(19) United States
(12) Patent Application Publication
(10) Pub. No.: US 2023/0157525 A1
(43) Pub. Date: May 25, 2023

(54) SYSTEM AND METHOD FOR REVERSING ORIENTATION AND VIEW OF SELECTED COMPONENTS OF A MINIATURIZED SURGICAL ROBOTIC UNIT IN VIVO

(71) Applicant: Vicarious Surgical Inc., Waltham, MA (US)

(72) Inventors: Banks Hunter, Cambridge, MA (US); Ryan Fish, Allston, MA (US); Sammy Khalifa, Medford, MA (US)

(21) Appl. No.: 18/095,315

(22) Filed: Jan. 10, 2023

Related U.S. Application Data
(63) Continuation of application No. PCT/US2021/031747, filed on May 11, 2021.
(60) Provisional application No. 63/023,034, filed on May 11, 2020.

(51) Int. Cl. A61B 1/00; A61B 1/045; A61B 34/32

(52) U.S. Cl. CPC A61B 1/00149; A61B 1/0016; A61B 1/045; A61B 34/32; A61B 2034/302

(57) ABSTRACT

A system and method for moving a robotic unit in vivo. The robotic unit can include a camera subassembly having a camera assembly coupled to a camera axially extending support member, a first robot arm subassembly having a first robot arm coupled to a first robot arm axially extending support member, and a second robot arm subassembly having a second robot arm axially extending support member, wherein when inserted in a cavity of a patient through an insertion point, the camera assembly and the first and second robot arms can be controlled for actuating at least one joint of each of the robot arms to reverse direction such that an end effector region of each of the first and second robot arms is facing towards the insertion point, and moving the camera assembly in a selected direction such that the camera elements are facing towards the insertion point.

### RELATED APPLICATION

This application is a 35 § U.S.C. 111(a) continuation application which claims the benefit of priority to PCT/US2021/031747, filed on May 11, 2021, which, in turn, claims the benefit of priority to U.S. provisional Pat. Application Serial No. 63/023,034, filed on May 11, 2020. The entire contents of each of the foregoing applications are incorporated herein by reference.

### BACKGROUND OF THE INVENTION

Since its inception in the early 1990s, the field of minimally invasive surgery has rapidly grown. While minimally invasive surgery vastly improves patient outcome, this improvement comes at a cost to the surgeon’s ability to operate with precision and ease. During conventional laparoscopic procedures, the surgeon typically inserts a laparoscopic instrument through multiple small incisions in the patient’s abdominal wall. The nature of tool insertion through the abdominal wall constrains the motion of the laparoscopic instruments as the instruments are unable to move side-to-side without injury to the abdominal wall. Standard laparoscopic instruments are also limited in motion, and are typically limited to four axes of motion. These four axes of motion are movement of the instrument in and out of the trocar (axis 1), rotation of the instrument within the trocar (axis 2), and angular movement of the trocar in two planes while maintaining the pivot point of the trocar’s entry into the abdominal cavity (axes 3 and 4). For over two decades, the majority of minimally invasive surgery has been performed with only these four degrees of motion. Moreover, prior systems require multiple incisions if the surgery requires addressing multiple different locations within the abdominal cavity.

Existing robotic surgical devices attempted to solve many of these problems. Some existing robotic surgical devices replicate non-robotic laparoscopic surgery with additional degrees of freedom at the end of the instrument. However, even with many costly changes to the surgical procedure, existing robotic surgical devices have failed to provide improved patient outcome in the majority of procedures for which they are used. Additionally, existing robotic devices create increased separation between the surgeon and surgical end-effectors. This increased separation causes injuries resulting from the surgeon’s misunderstanding of the motion and the force applied by the robotic device. Because the degrees of freedom of many existing robotic devices are unfamiliar to a human operator, surgeons need extensive training on robotic simulators before operating on a patient in order to minimize the likelihood of causing inadvertent injury.

To control existing robotic devices, a surgeon typically sits at a console and controls manipulators with his or her hands and/or feet. Additionally, robot cameras remain in a semi-fixed location, and are moved by a combined foot and hand motion from the surgeon. These semi-fixed cameras offer limited fields of view and often result in difficulty visualizing the operating field.

Other robotic devices have two robotic manipulators inserted through a single incision. These devices reduce the number of incisions required to a single incision, often in the umbilicus. However, existing single-incision robotic devices have significant shortcomings stemming from their actuator design. Existing single-incision robotic devices include servomotors, encoders, gearboxes, and all other actuation devices within the in vivo robot, which results in relatively large robotic units that are inserted within the patient. This size severely constrains the robotic unit in terms of movement and ability to perform various procedures. Further, such a large robot typically needs to be inserted through a large incision site, oftentimes near the size of open surgery, thus increasing risk of infection, pain, and general morbidity.

A further drawback of conventional robotic devices is their limited degrees of freedom of movement. Hence, if the surgical procedure requires surgery at multiple different locations, then multiple incision points need to be made so as to be able to insert the robotic unit at the different operating locations. This increases the chance of infection of the patient.

### SUMMARY OF THE INVENTION

The present invention is directed to a surgical robotic system that employs a camera assembly having at least three articulating degrees of freedom and one or more robotic arms having at least six articulating degrees of freedom and an additional degree of freedom corresponding to the movement of an associated end-effector (e.g., grasper, manipulator, and the like). The camera assembly when mounted within the patient can be moved or rotated in a pitch or yaw direction about 180 degrees such that the camera assembly can view rearwardly back towards the insertion site. As such, the camera assembly and the robotic arms can view and operate dexterously forward (e.g., away from the insertion site), to each side, in an upward or downward direction, as well as in the rearward direction to view backwards towards the insertion site. The robot arms and the camera assembly can also move in the roll, pitch and yaw directions.

The present invention is also directed to a robot support system that includes a support stanchion that employs one or more adjustment elements and associated pivot joints. The motor unit of the robotic subsystem can be mounted to a distalmost one of the adjustment elements. The motor unit can employ multiple adjustment elements and pivot points for linearly or axially moving one or more components of the robotic unit, including for example the robot arms and the camera assembly.

The present invention is directed to a a surgical robotic system comprising a computing unit for receiving user generated movement data and for generating control signals in response thereto, a robot support subsystem having a support stanchion, and a robotic subsystem. The support stanchion includes a base portion, a support beam having a first end coupled to the base and an opposed second end coupled to a proximal one of a plurality of adjustment elements. The adjustment elements are arranged and disposed to form pivot joints between adjacent ones of the adjustment elements and between the proximal one adjustment element and the support beam. The robotic subsystem includes a motor unit having one or more motor elements associated therewith, where the motor unit is coupled to a distal one of the plurality of adjustment elements, and a robotic unit having a camera subassembly and a plurality of robot arm subassemblies. The camera subassembly and the plurality of robot arm subassemblies are coupled to the motor unit, and the motor unit when actuated moves one of the camera subassembly and the robot arm subassemblies in a selected direction. Further, one or more of the adjustment elements and one or more of the camera subassembly and the robot arm subassemblies move in response to the control signals.

The camera subassembly includes an axially extending support member, an interface element coupled to one end of the support member, and a camera assembly coupled to an opposed end of the support member. The interface element is configured for engaging with one or more of the motor elements of the motor unit. Further, the camera assembly includes a first camera element having a first light source associated therewith and a second camera element having a second light source associated therewith. The robot arm subassemblies include an axially extending support member, an interface element coupled to one end of the support member, and a robot arm coupled to an opposed end of the support member. Further, each of the interface elements of the robot arm subassemblies is configured for engaging with different one of a plurality of motor elements of the motor unit. The interface element of the camera subassembly can be coupled to the same motor element as the interface element of one of the robot arm subassemblies. Alternatively, the interface element of the camera subassembly and the interface element of one of the robot arm subassemblies can be coupled to different ones of the plurality of motor elements.

Further, the robot arms can include an end effector region and the camera assembly and the first and second robot arms can be sized and configured to be inserted into a cavity of a patient through an insertion point, and the computing unit in response to the user generated control signals can generate control signals which are received by the first and second robot arms and the camera assembly. In response to the control signals, each of the first and second robot arms can be actuated so as to reverse direction such that the end effector region is facing towards the insertion point, and the camera assembly can be moved in a selected direction such that the camera elements are facing towards the insertion point. Alternatively, in response to the control signals, the robot arms can be oriented or moved such that they face in a first direction that is transverse or orthogonal to an axis of the support member, and each of the first and second robot arms can be actuated so as to reverse direction such that the end effector region is facing in a second direction that is substantially opposite the first direction. Still further, in response to the control signals, the robot arms can be oriented such that they face in a first direction, and each of the first and second robot arms can be actuated or moved so as to reverse direction such that the end effector region is facing in a second direction that is substantially opposite the first direction.

According to the present invention, prior to moving the camera assembly towards the insertion point, the camera support member can be rotated so that the camera assembly is disposed above the camera support member and one or more camera elements of the camera assembly are facing away from the insertion point. Further, the camera assembly can be rotated in a pitch direction such that the camera elements are facing towards the insertion point. Alternatively, the camera assembly can be rotated in a yaw direction such that the camera elements are facing towards the insertion point.

The present invention is also directed to a method for moving a robotic unit in vivo. The robotic unit can include a camera subassembly having a camera assembly coupled to a camera axially extending support member, a first robot arm subassembly having a first robot arm coupled to a first robot arm axially extending support member, and a second robot arm subassembly having a second robot arm axially extending support member, wherein when inserted in a cavity of a patient through an insertion point, the camera assembly and the first and second robot arms can be controlled for actuating at least one joint of each of the robot arms to reverse direction such that an end effector region of each of the first and second robot arms is facing towards the insertion point, and moving the camera assembly in a selected direction such that the camera elements are facing towards the insertion point.

The robotic unit can be connected to a motor unit and the motor unit can be actuated or driven so as to move the robotic unit or the camera assembly relative to the insertion site in a translational or linear direction. Each of the interface elements of the first and second robot arm subassemblies can be configured for engaging with different ones of a plurality of motor elements of the motor unit. Alternatively, the interface element of the camera subassembly can be coupled to the same motor element as the interface element of one of the first and second robot arm subassemblies. Further, the interface element of the camera subassembly and the interface element of one of the first and second robot arm subassemblies can be coupled to different ones of the plurality of motor elements.

According to the method of the present invention, prior to moving the camera assembly, the camera support member can be rotated so that the camera assembly is disposed above the camera support member and one or more camera elements of the camera assembly are facing away from the insertion point. The step of moving the camera assembly can comprise rotating the camera assembly in a pitch direction such that the camera elements are facing towards the insertion point. Alternatively, the step of moving the camera assembly can include rotating the camera assembly in a yaw direction such that the camera elements are facing towards the insertion point.

The present invention can also be directed to a method for moving a robotic unit in vivo, where the robotic unit includes a camera subassembly having a camera assembly coupled to a camera axially extending support member, a first robot arm subassembly having a first robot arm coupled to a first robot arm axially extending support member, and a second robot arm subassembly having a second robot arm coupled to an axially extending support member, wherein when inserted in a cavity of a patient through an insertion point. The camera assembly and the first and second robot arms can be controlled for actuating at least one joint on each of the first and second robot arms to reverse direction such that each an end-effector region of each of the first and second robot arms is facing in a direction that is orthogonal to an insertion axis, and actuating at least one joint of the camera assembly to move the camera assembly in a selected direction such that the camera elements are facing in a direction orthogonal to the insertion axis.

When the robotic unit is connected to a motor unit, the method includes actuating the motor unit so as to move the robotic unit or the camera assembly relative to the insertion site. Further, each of the interface elements of the first and second robot arm subassemblies is configured for engaging with a different one of the motor elements of the motor unit. Alternatively, the interface element of the camera subassembly is coupled to the same motor element as the interface element of one of the first and second robot arm subassemblies. Further, the interface element of the camera subassembly and the interface element of one of the first and second robot arm subassemblies are coupled to different ones of the plurality of motor elements.

The method also includes, prior to moving the camera assembly, rotating the camera support member so that the camera assembly is disposed above the camera support member and one or more camera elements of the camera assembly are facing away from the reverse facing direction. The step of moving the camera assembly includes rotating the camera assembly in a pitch or yaw direction such that the camera elements are facing in the reverse facing direction.

### BRIEF DESCRIPTION OF THE DRAWINGS

These and other features and advantages of the present invention will be more fully understood by reference to the following detailed description in conjunction with the attached drawings in which like reference numerals refer to like elements throughout the different views. The drawings illustrate principals of the invention and, although not to scale, show relative dimensions.

FIG. 1 is a schematic illustration of the surgical robotic system of the present invention.

FIG. 2A is a perspective view of a robot arm subassembly according to the teachings of the present invention.

FIG. 2B is a perspective view of a camera subassembly according to the teachings of the present invention.

FIG. 3A is a perspective side view of a support stanchion that forms part of a robotic support system employed by the surgical robotic system that is coupled to a robotic subsystem according to the teachings of the present invention.

FIGS. 3B-3D are perspective side views of the support stanchion coupled to the motor unit of the robotic subsystem, where the motor unit employs multiple motor elements, and where the motor elements are coupled to the camera subassembly and the robot arm subassemblies according to the teachings of the present invention.

FIGS. 3E-3G are perspective top views of the support stanchion coupled to the motor unit of the robotic subsystem, where the motor unit employs multiple motor elements, and where the motor elements are coupled to the camera subassembly and the robot arm subassemblies according to the teachings of the present invention.

FIG. 4 is a pictorial perspective view of robotic unit disposed within a body cavity of a patient according to the teachings of the present invention.

FIG. 5 is a pictorial perspective view of robotic unit disposed within a body cavity of a patient where the robot arms and camera assembly are disposed in a neutral position according to the teachings of the present invention.

FIG. 6A is a pictorial perspective view of the robotic unit disposed within the body cavity of the patient where the robot arms are shown moving towards a rear facing position according to the teachings of the present invention.

FIG. 6B is a pictorial perspective view of the robotic unit disposed within the body cavity of the patient where the camera subassembly is shown moving in a roll direction according to the teachings of the present invention.

FIG. 6C is a pictorial perspective view of the robotic unit disposed within the body cavity of the patient where the camera assembly is shown moving in a pitch direction so as to be rearward facing according to the teachings of the present invention.

FIG. 6D is a pictorial perspective view of the robotic unit disposed within the body cavity of the patient where the camera assembly is shown moving in an alternate yaw direction so as to be rearward facing according to the teachings of the present invention.

FIG. 7A is a perspective view of an alternate embodiment of the camera subassembly of the surgical robotic system of the present invention.

FIG. 7B is a partial view of the camera assembly of FIG. 7A illustrating the rotational axes that are implemented by the articulation joints according to the teachings of the present invention.

FIG. 8A is a perspective view of another embodiment of the camera subassembly of the present invention.

FIG. 8B is a perspective view of the camera assembly of FIG. 8A disposed in an articulated position.

### DETAILED DESCRIPTION

The present invention employs a surgical robotic unit that can be inserted into a patient via a trocar through a single incision point or site. The robotic unit is small enough to be deployed in vivo at the surgical site, and is sufficiently maneuverable when inserted to be able to move within the body so as to perform various surgical procedures at multiple different points or sites. Specifically, the robotic unit can be inserted and the camera assembly and robotic arms controlled and manipulated so that they are oriented backward in a rear facing direction. Further, the robotic subsystem can be coupled to a support stanchion that forms part of a robotic support system. The support stanchion can have multiple adjustment or articulating sections so that they can impart, when properly manipulated and oriented, linear movement to one or more components of the robotic unit.

In the following description, numerous specific details are set forth regarding the system and method of the present invention and the environment in which the system and method may operate, in order to provide a thorough understanding of the disclosed subject matter. It will be apparent to one skilled in the art, however, that the disclosed subject matter may be practiced without such specific details, and that certain features, which are well known in the art, are not described in detail in order to avoid complication and enhance clarity of the disclosed subject matter. In addition, it will be understood that any examples provided below are merely illustrative and are not to be construed in a limiting manner, and that it is contemplated by the present inventors that other systems, apparatuses, and/or methods can be employed to implement or complement the teachings of the present invention and are deemed to be within the scope of the present invention.

While the system and method of the present invention can be designed for use with one or more surgical robotic systems employed as part of a virtual reality surgical system, the robotic system of the present invention may be employed in connection with any type of surgical system, including for example robotic surgical systems, straight-stick type surgical systems, and laparoscopic systems. Additionally, the system of the present invention may be used in other non-surgical systems, where a user requires access to a myriad of information, while controlling a device or apparatus.

The system and method disclosed herein can be incorporated and utilized with the robotic surgical device and associated system disclosed for example in U.S. Pat. No. 10,285,765 and in PCT patent application Serial No. PCT/US20/39203, and/or with the camera system disclosed in U.S. Publication No. 2019/0076199, where the content and teachings of all of the foregoing patents, patent applications and publications are herein incorporated by reference. The surgical robotic unit that forms part of the present invention can for part of a surgical system that includes a user workstation, a robot support system (RSS) for interacting with and supporting the robotic subsystem, a motor unit, and an implantable surgical robotic unit that includes one or more robot arms and one or more camera assemblies. The implantable robot arms and camera assembly can form part of a single support axis robotic system or can form part of a split arm (SA) architecture robotic system.

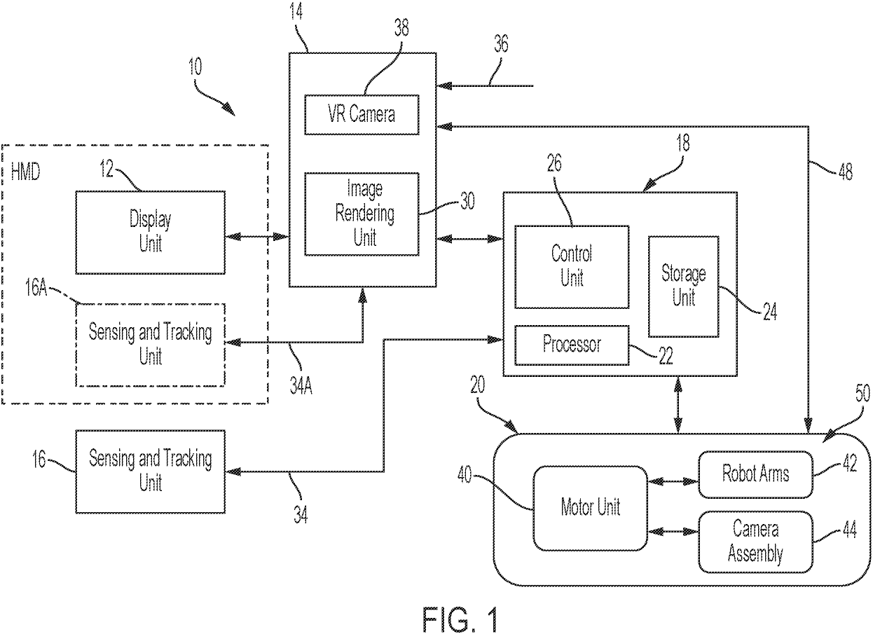

FIG. 1 is a schematic block diagram description of a surgical robotic system 10 according to the teachings of the present invention. The system 10 includes a display device or unit 12, a virtual reality (VR) computing unit 14, a sensing and tracking unit 16, a computing unit 18, and a robotic subsystem 20. The display unit 12 can be any selected type of display for displaying information, images or video generated by the VR computing unit 14, the computing unit 18, and/or the robotic subsystem 20. The display unit 12 can include or form part of for example a head-mounted display (HMD), a screen or display, a three-dimensional (3D) screen, and the like. The display unit can also include an optional sensor and tracking unit 16A, such as can be found in commercially available head mounted displays. The sensing and tracking units 16 and 16A can include one or more sensors or detectors that are coupled to a user of the system, such as for example a nurse or a surgeon. The sensors can be coupled to the arms of the user and if a head-mounted display is not used, then additional sensors can also be coupled to a head and/or neck region of the user. The sensors in this arrangement are represented by the sensor and tracking unit 16. If the user employs a head-mounted display, then the eyes, head and/or neck sensors and associated tracking technology can be built-in or employed within that device, and hence form part of the optional sensor and tracking unit 16A. The sensors of the sensor and tracking unit 16 that are coupled to the arms of the surgeon can be preferably coupled to selected regions of the arm, such as for example the shoulder region, the elbow region, the wrist or hand region, and if desired the fingers. According to one practice, the sensors are coupled to a pair of hand controllers that are manipulated by the surgeon. The sensors generate position data indicative of the position of the selected portion of the user. The sensing and tracking units 16 and/or 16A can be utilized to control movement of the camera assembly 44 and the robotic arms 42 of the robotic subsystem 20. The position data 34 generated by the sensors of the sensor and tracking unit 16 can be conveyed to the computing unit 18 for processing by a processor 22. The computing unit 20 can determine or calculate from the position data 34 the position and/or orientation of each portion of the surgeon’s arm and convey this data to the robotic subsystem 20. According to an alternate embodiment, the sensing and tracking unit 16 can employ sensors coupled to the torso of the surgeon or any other body part. Further, the sensing and tracking unit 16 can employ in addition to the sensors an Inertial Momentum Unit (IMU) having for example an accelerometer, gyroscope, magnetometer, and a motion processor. The addition of a magnetometer is standard practice in the field as magnetic heading allows for reduction in sensor drift about the vertical axis. Alternate embodiments also include sensors placed in surgical material such as gloves, surgical scrubs, or a surgical gown. The sensors may be reusable or disposable. Further, sensors can be disposed external of the user, such as at fixed locations in a room, such as an operating room. The external sensors can generate external data 36 that can be processed by the computing unit and hence employed by the system 10. According to another embodiment, when the display unit 12 is a head mounted device that employs an associated sensor and tracking unit 16A, the device generates tracking and position data 34A that is received and processed by the VR computing unit 14. Further, the sensor and tracking unit 16 can include if desired a hand controller.

In the embodiment where the display is a HMD, the display unit 12 can be a virtual reality head-mounted display, such as for example the Oculus Rift, the Varjo VR-1 or the HTC Vive Pro Eye. The HMD can provide the user with a display that is coupled or mounted to the head of the user, lenses to allow a focused view of the display, and a sensor and/or tracking system 16A to provide position and orientation tracking of the display. The position and orientation sensor system can include for example accelerometers, gyroscopes, magnetometers, motion processors, infrared tracking, eye tracking, computer vision, emission and sensing of alternating magnetic fields, and any other method of tracking at least one of position and orientation, or any combination thereof. As is known, the HMD can provide image data from the camera assembly 44 to the right and left eyes of the surgeon. In order to maintain a virtual reality experience for the surgeon, the sensor system can track the position and orientation of the surgeon’s head, and then relay the data to the VR computing unit 14, and if desired to the computing unit 18. The computing unit 18 can further adjust the pan and tilt of the camera assembly 44 of the robot so as to follow the movement of the user’s head.

The sensor or position data 34A generated by the sensors if associated with the HMD, such as for example associated with the display unit 12 and/or tracking unit 16A, can be conveyed to the computing unit 18 either directly or via the VR computing unit 14. Likewise, the tracking and position data 34 generated by the other sensors in the system, such as from the sensing and tracking unit 16 that can be associated with the user’s arms and hands, can be conveyed to the computing unit 18. The tracking and position data 34, 34A can be processed by the processor 22 and can be stored for example in the storage unit 24. The tracking and position data 34, 34A can also be used by the control unit 26, which in response can generate control signals for controlling movement of one or more portions of the robotic subsystem 20. The robotic subsystem 20 can include a user workstation, the robot support system (RSS), a motor unit 40, and an implantable surgical robot unit that includes one or more robot arms 42 and one or more camera assemblies 44. The implantable robot arms and camera assembly can form part of a single support axis robot system, such as that disclosed and described in U.S. Pat. No. 10,285,765, or can form part of a split arm (SA) architecture robot system, such as that disclosed and described in PCT patent application no. PCT/US20/39203.

The control signals generated by the control unit 26 can be received by the motor unit 40 of the robotic subsystem 20. The motor unit 40 can include a series of servomotors and gears that are configured for driving separately the robot arms 42 and the cameras assembly 44. The robot arms 42 can be controlled to follow the scaled-down movement or motion of the surgeon’s arms as sensed by the associated sensors. The robot arms 42 can have portions or regions that can be associated with movements associated with the shoulder, elbow, and wrist joints as well as the fingers of the user. For example, the robotic elbow joint can follow the position and orientation of the human elbow, and the robotic wrist joint can follow the position and orientation of the human wrist. The robot arms 42 can also have associated therewith end regions that can terminate in end-effectors that follow the movement of one or more fingers of the user, such as for example the index finger as the user pinches together the index finger and thumb. While the arms of the robot follow movement of the arms of the user, the robot shoulders are fixed in position. In one embodiment, the position and orientation of the torso of the user is subtracted from the position and orientation of the users arms. This subtraction allows the user to move his or her torso without the robot arms moving.

The robot camera assembly 44 is configured to provide the surgeon with image data 48, such as for example a live video feed of an operation or surgical site, as well as enable a surgeon to actuate and control the cameras forming part of the camera assembly 44. The camera assembly 44 preferably includes a pair of cameras 70A, 70B, the optical axes of which are axially spaced apart by a selected distance, known as the inter-camera distance, so as to provide a stereoscopic view or image of the surgical site. The surgeon can control the movement of the cameras 70A, 70B either through movement of a head-mounted display or via sensors coupled to the head of the surgeon, or by using a hand controller or sensors tracking the user’s head or arm motions, thus enabling the surgeon to obtain a desired view of an operation site in an intuitive and natural manner. The cameras are movable in multiple directions, including for example in the yaw, pitch and roll directions, as is known. The components of the stereoscopic cameras can be configured to provide a user experience that feels natural and comfortable. In some embodiments, the interaxial distance between the cameras can be modified to adjust the depth of the operation site perceived by the user.

According to one embodiment, the camera assembly 44 can be actuated by movement of the surgeon’s head. For example, during an operation, if the surgeon wishes to view an object located above the current field of view (FOV), the surgeon looks in the upward direction, which results in the stereoscopic cameras being rotated upward about a pitch axis from the user’s perspective. The image or video data 48 generated by the camera assembly 44 can be displayed on the display unit 12. If the display unit 12 is a head-mounted display, the display can include the built-in tracking and sensor system 16A that obtains raw orientation data for the yaw, pitch and roll directions of the HMD as well as positional data in Cartesian space (x, y, z) of the HMD. However, alternative tracking systems may be used to provide supplementary position and orientation tracking data of the display in lieu of or in addition to the built-in tracking system of the HMD.

The image data 48 generated by the camera assembly 44 can be conveyed to the virtual reality (VR) computing unit 14 and can be processed by the VR or image rendering unit 30. The image data 48 can include still photographs or image data as well as video data. The VR rendering unit 30 can include suitable hardware and software for processing the image data and then rendering the image data for display by the display unit 12, as is known in the art. Further, the VR rendering unit 30 can combine the image data received from the camera assembly 44 with information associated with the position and orientation of the cameras in the camera assembly, as well as information associated with the position and orientation of the head of the surgeon. With this information, the VR rendering unit 30 can generate an output video or image rendering signal and transmit this signal to the display unit 12. That is, the VR rendering unit 30 renders the position and orientation readings of the hand controllers and the head position of the surgeon for display in the display unit, such as for example in a HMD worn by the surgeon.

The VR computing unit 14 can also include a virtual reality (VR) camera unit 38 for generating one or more virtual reality (VR) cameras for use or emplacement in the VR world that is displayed in the display unit 12. The VR camera unit 38 can generate one or more virtual cameras in a virtual world, and which can be employed by the system 10 to render the images for the head-mounted display. This ensures that the VR camera always renders the same views that the user wearing the head-mounted display sees to a cube map. In one embodiment, a single VR camera can be used and in another embodiment separate left and right eye VR cameras can be employed to render onto separate left and right eye cube maps in the display to provide a stereo view. The FOV setting of the VR camera can self-configure itself to the FOV published by the camera assembly 44. In addition to providing a contextual background for the live camera views or image data, the cube map can be used to generate dynamic reflections on virtual objects. This effect allows reflective surfaces on virtual objects to pick up reflections from the cube map, making these objects appear to the user as if they’re actually reflecting the real world environment.

The robotic subsystem 20 can employ multiple different robotic arms 42A, 42B that are deployable along different or separate axes. Further, the camera assembly 44, which can employ multiple different camera elements 70A, 70B, can also be deployed along a common separate axis. Thus, the surgical robotic unit employs multiple different components, such as a pair of separate robotic arms and a camera assembly 44, which are deployable along different axes. Further, the robot arms 42 and the camera assembly 44 are separately manipulatable, maneuverable, and movable. The robotic subsystem 20, which includes the robot arms and the camera assembly, is disposable along separate manipulatable axes, and is referred to herein as a Split Arm (SA) architecture. The SA architecture is designed to simplify and increase efficiency of the insertion of robotic surgical instruments through a single trocar at a single insertion point or site, while concomitantly assisting with deployment of the surgical instruments into a surgical ready state, as well as the subsequent removal of the surgical instruments through the trocar. By way of example, a surgical instrument can be inserted through the trocar to access and perform an operation in vivo in a body cavity of a patient. In some embodiments, various surgical instruments may be utilized, including but not limited to robotic surgical instruments, as well as other surgical instruments known in the art.

In some embodiments, the robotic subsystem 20 of the present invention is supported by a structure with multiple degrees of freedom such that the robotic arms 42A, 42B and camera assembly 44 (e.g., robotic unit 50) can be maneuvered within the patient into a single position or multiple different positions. In some embodiments, the robotic subsystem 20 can be directly mounted to a surgical table or to the floor or ceiling within an operating room, or to any other types of support structure. In other embodiments, the mounting is achieved by various fastening means, including but not limited to clamps, screws, or a combination thereof. In still further embodiments, the support structure may be free standing. The support structure is referred to herein as the robot support system (RSS). The RSS can form part of an overall surgical robotic system 10 that can include a virtual station that allows a surgeon to perform virtual surgery within the patient.

In some embodiments, the RSS of the surgical robotic system 10 can optionally include the motor unit 40 that is coupled to the robotic unit 50 at one end and to an adjustable support member or element at an opposed end. Alternatively, as shown herein, the motor unit 40 can form part of the robotic subsystem 20. The motor unit 40 can include gears, one or more motors, drivetrains, electronics, and the like, for powering and driving one or more components of the robotic unit 50. The robotic unit 50 can be selectively coupled to the motor unit 40. According to one embodiment, the RSS can include a support member that has the motor unit 40 coupled to a distal end thereof. The motor unit 40 in turn can be coupled to the camera assembly 44 and to each of the robot arms 42. The support member can be configured and controlled to move linearly, or in any other selected direction or orientation, one or more components of the robotic unit 50.

The motor unit 40 can also provide mechanical power, electrical power, mechanical communication, and electrical communication to the robotic unit 50, and can further include an optional controller for processing input data from one or more of the system components (e.g., the display 12, the sensing and tracking unit 16, the robot arms 42, the camera assembly 44, and the like), and for generating control signals in response thereto. The motor unit 40 can also include a storage element for storing data. Alternatively, the motor unit 40 can be controlled by the computing unit 18. The motor unit 40 can thus generate signals for controlling one or more motors that in turn can control and drive the robot arms 42, including for example the position and orientation of each articulating joint of each arm, as well as the camera assembly 44. The motor unit 40 can further provide for a translational or linear degree of freedom that is first utilized to insert and remove each component of the robotic unit 50 through a suitable medical device, such as a trocar 108. The motor unit 40 can also be employed to adjust the inserted depth of each robot arm 42 when inserted into the patient 100 through the trocar 108.

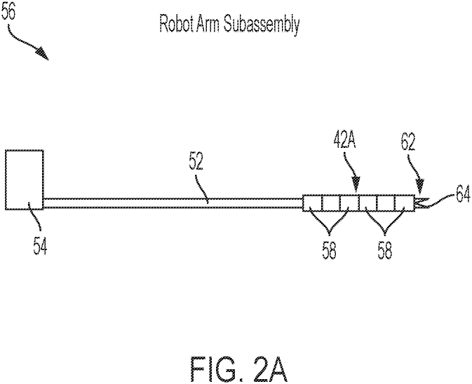

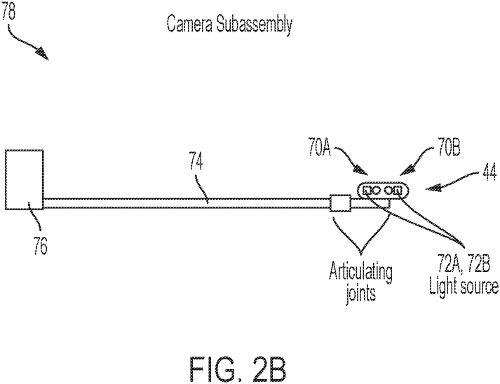

FIGS. 2A and 2B illustrate the general design of selected components of the robotic subsystem 20 of the present invention. For example, FIG. 2A illustrates the robot arm subassembly 56 of the present invention. The illustrated robot arm subassembly 56 includes an axially extending support member 52 that has an interface element 54 coupled to a proximal end and a robot arm 42A coupled to an opposed distal end. The support member 52 serves to support the robot arm 42A when mounted thereto, and can further function as a conduit for mechanical power, electrical power, and communication. For the sake of simplicity, only the first robot arm 42A is shown, although the second robot arm 42B or subsequent arms can be similar or identical. The interface element 54 is configured to connect to the motor unit 40 for transferring a driving force therefrom and any associated signals, via the support element 52, to the robot arm 42A. The interface element can have any selected shape and size is preferably configured to engage with a driving end of a motor element of the motor unit 40. In one embodiment, the interface element 54, 76 can employ a series of electrical contacts and a series of mechanical linkage devices, such as pulleys, each having a rotational axis. In another embodiment, the mechanical pulleys can each include a male spline protruding from the surface of the interface element. Each male spline is configured to mate with a female spline located on the drive element and thus provide for the transmission of mechanical power in the form of torque. In still another embodiment, the pulleys can employ one or more female splines that engage with one or more male splines that are located on the drive elements. In still other embodiments, the mechanical power from the drive elements can be transferred to the interface elements by other mating type surfaces as is known in the art. Further, the illustrated robot arm 42A can include a series of articulation sections 58 that form joint sections that correspond to the joints of a human arm. As such, the articulation sections 58 can be constructed and combined to provide for rotational and/or hinged movement so as to emulate different portions of the human arm, such as for example the shoulder j oint or region, elbow j oint or region, and the wrist j oint or region. The articulation sections 58 of the robot arm 42A are constructed to provide cable-driven, rotational movement, for example, but within the confines of reasonable rotational limits. The articulation sections 58 are configured to provide maximum torque and speed with minimum size. In an alternate embodiment, the articulation sections 58 can include spherical joints, thus providing for multiple, such as two or three, rotational degrees of freedom in a single joint.

In one embodiment, each articulation section 58 can be oriented orthogonally, relative to a starting point, to an adjacent articulation section. Further, each articulation section 58 can be cable driven and can have a Hall Effect sensor array associated therewith for joint position tracking. In another embodiment, the articulation section can include inertial measurement units or magnetic tracking solutions, such as those provided by Polhemus, USA, that are integrated therein so as to provide for joint position tracking or estimation. Further, communication wires for the sensors as well as the mechanical drive cables can be routed proximally through an inner chamber of the support member 52 to the proximal interface element 54. The robot arm 42A can also include an end portion 62 that can have coupled thereto one or more surgical tools, as is known in the art. According to one embodiment, an end effector or grasper 64 can be coupled to the end portion 62. The end effector can mimic movement of one or more of the surgeon’s fingers.

FIG. 2B illustrates the camera subassembly 78 of the present invention. The illustrated camera assembly can include an axially extending support member 74 that has an interface element 76 coupled to a proximal end and a camera assembly 44 coupled to an opposed distal end. The illustrated camera assembly 44 can include a pair of camera elements 70A, 70B. The camera assembly can be connected or coupled to the support member 74 in such a manner so as to allow movement of the camera assembly relative to the support member in the yaw and pitch directions. The camera elements can be separate and distinct relative to each other or can be mounted in a common housing, as shown. Each of the camera elements 70A, 70B can have a light source 72A, 72B, respectively, associated therewith. The light source can be disposed at any selected location relative to the camera elements. The support member 74 serves to support the camera assembly 44 when mounted thereto, and can further function as a conduit for mechanical power, electrical power, and communication. The interface element 76 is configured to connect to the motor unit 40 for transferring a driving force therefrom and any associated signals, via the support element 52, to the camera assembly 44.

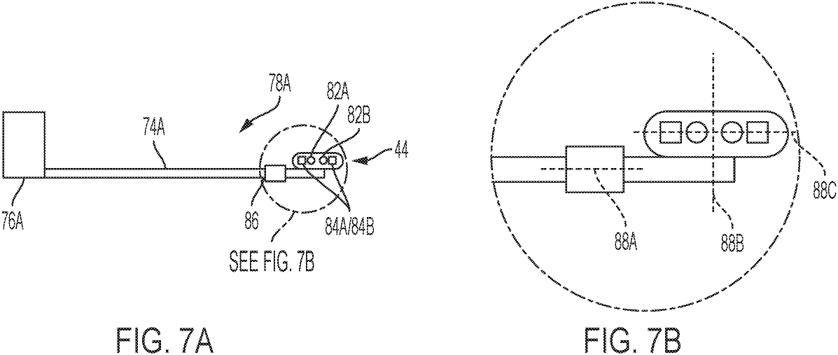

An alternate embodiment of the camera subassembly of the present invention is shown in FIGS. 7A and 7B. The illustrated camera subassembly 78A can include an axially extending support member 74A that has an interface element 76A coupled to a proximal end and a camera assembly 44 coupled to an opposed distal end. The illustrated camera assembly 44 can include a pair of camera elements 82A, 82B. The camera assembly 44 can be connected or coupled to the support member 74A in such a manner so as to allow for movement of the camera assembly relative to the support member. The camera elements 82A, 82B can be separate and distinct relative to each other or can be mounted in a common housing, as shown. Each of the camera elements can have a light source 84A, 84B, respectively, associated therewith. The light sources 84A, 84B can be disposed at any selected location relative to the camera elements. The support member 74A serves to support the camera assembly 44 when mounted thereto, and can further function as a conduit for mechanical power, electrical power, and communication. The interface element 76A is configured to connect to the motor unit 40 for transferring a driving force therefrom and any associated signals, via the support element 52, to the camera assembly 44. The illustrated support member 74A can also include one or more articulating joints 86 that allow for movement of the camera assembly 44 relative to the support member 74A in multiple degrees of freedom, including for example in three degrees of freedom. The multiple degrees of freedom of the camera assembly 44 can be implemented by the articulating joints 86. The multiple degrees of freedom can include for example movement about a roll axis 88A, a yaw axis 88B, and a pitch axis 88C, as shown in FIG. 7B.

The articulating joints 86 can include for example a series of sequential hinge joints each of which is orthogonal to the adjacent or previous joint, and the camera assembly 44 can be coupled to the distalmost articulating joint 86. This arrangement forms in essence a snake-like camera subassembly that can is capable of actuating the articulating joints 86 such that the camera assembly 44 can be repositioned and angled to view a relatively large portion of the body cavity. Further, one of the degrees of freedom can include a rotational degree of freedom, the axis of which is parallel to a lengthwise axis of the support member 74A. This additional axis is also orthogonal to the other axes and can provide for increased maneuverability to the camera subassembly. Further, the maneuverability and position ability of the camera subassembly can be enhanced by adding greater than three degrees of freedom. In some embodiments, the illustrated camera subassembly 78A can include a series of spherical or ball-like joints, each individually enabling two or three degrees of freedom. The ball joints can enable similar degrees of freedom in a smaller package.

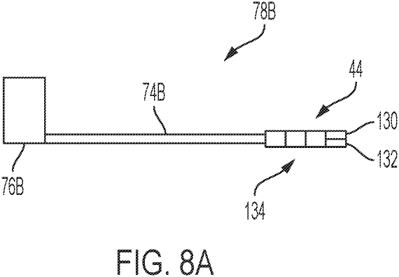

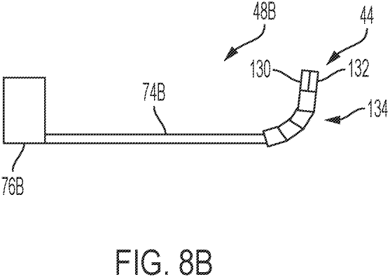

Still another embodiment of the camera subassembly is shown in FIGS. 8A and 8B. The illustrated camera subassembly 78B can include an axially extending support member 74B that has an interface element 76B coupled to a proximal end and a camera assembly 44 coupled to an opposed distal end. The illustrated camera assembly 44 can be differently configured and can include for example a stacked assembly including an imaging unit 130 having a pair of camera elements and a light unit 132 that includes one or more light sources. The camera assembly 44 can be connected or coupled to the support member 74B in such a manner so as to allow for movement of the camera assembly relative to the support member. The support member 74B serves to support the camera assembly 44 when mounted thereto, and can further function as a conduit for mechanical power, electrical power, and communication. The interface element 76B is configured to connect to the motor unit 40 for transferring a driving force therefrom and any associated signals, via the support element 52, to the camera assembly 44. The illustrated support member 74B can also include one or more articulating joints 134 that allow for movement of the camera assembly 44 relative to the support member 74A in multiple degrees of freedom, including for example in three degrees of freedom. The multiple degrees of freedom of the camera assembly 44 can be implemented by the articulating joints 134. The camera assembly 44 can be moved using the articulating joints, similar to the camera subassembly 78A. FIG. 8B shows the distal end of the camera subassembly disposed in a bent articulated position.

The robot arm subassemblies 56, 56 and the camera subassembly 78 are capable of multiple degrees of freedom of movement. According to one practice, when the robot arm assemblies 56, 56 and the camera subassembly 78 are inserted into a patient through a trocar, the subassemblies are capable of movement in at least the axial, yaw, pitch, and roll directions. The robot arm assemblies 56, 56 are configured to incorporate and utilize multiple degrees of freedom of movement with an optional end effector 64 mounted at a distal end thereof. In other embodiments, the working or distal end of the robot arm assemblies 56, 56 is designed to incorporate and utilize other robotic surgical instruments.

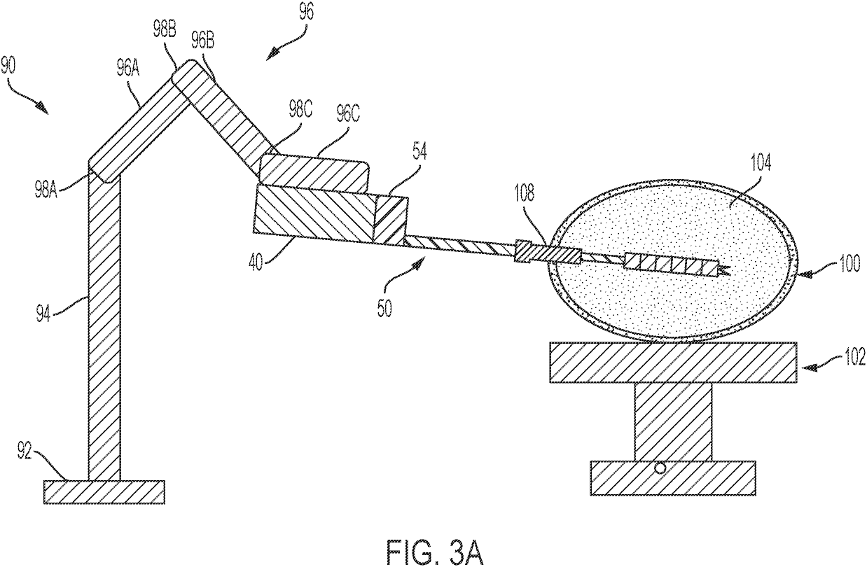

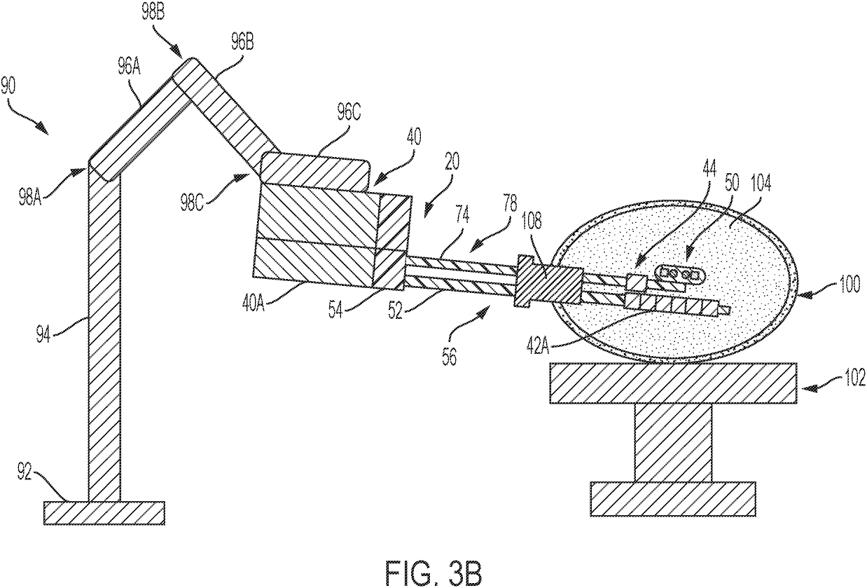

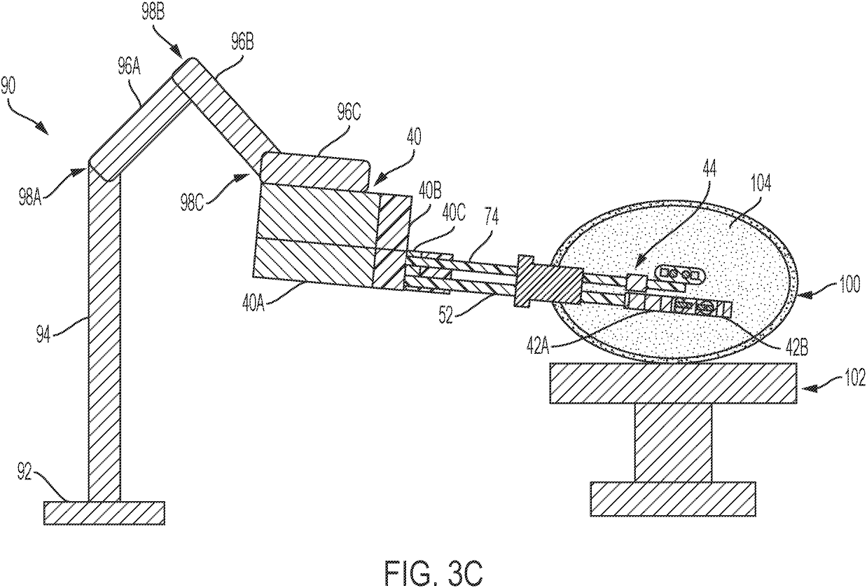

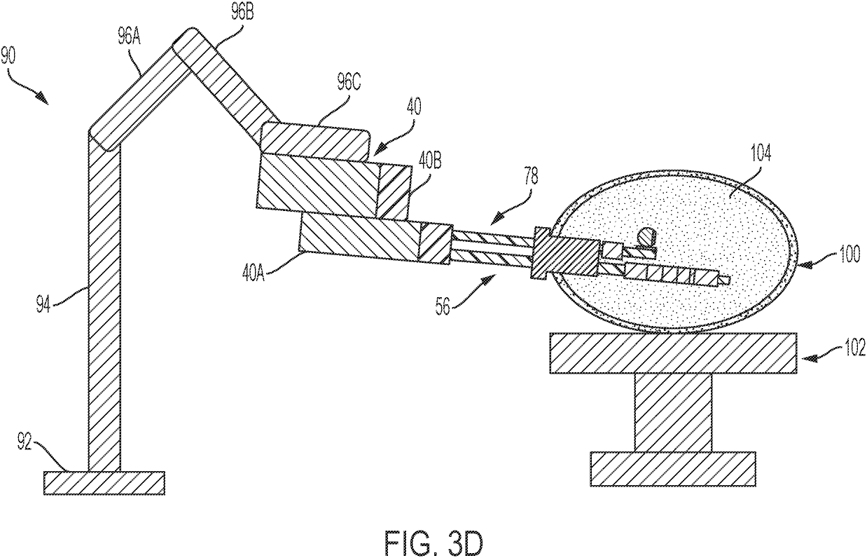

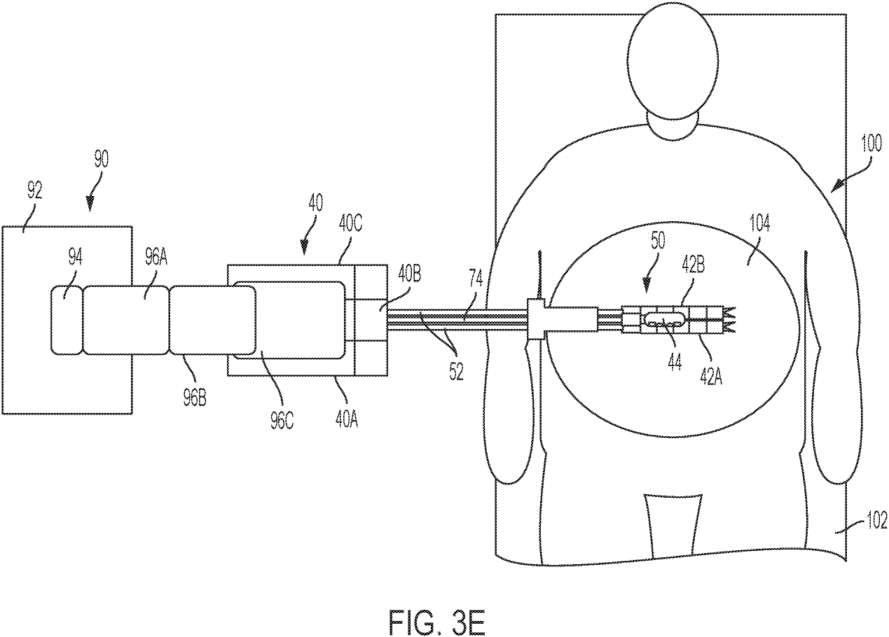

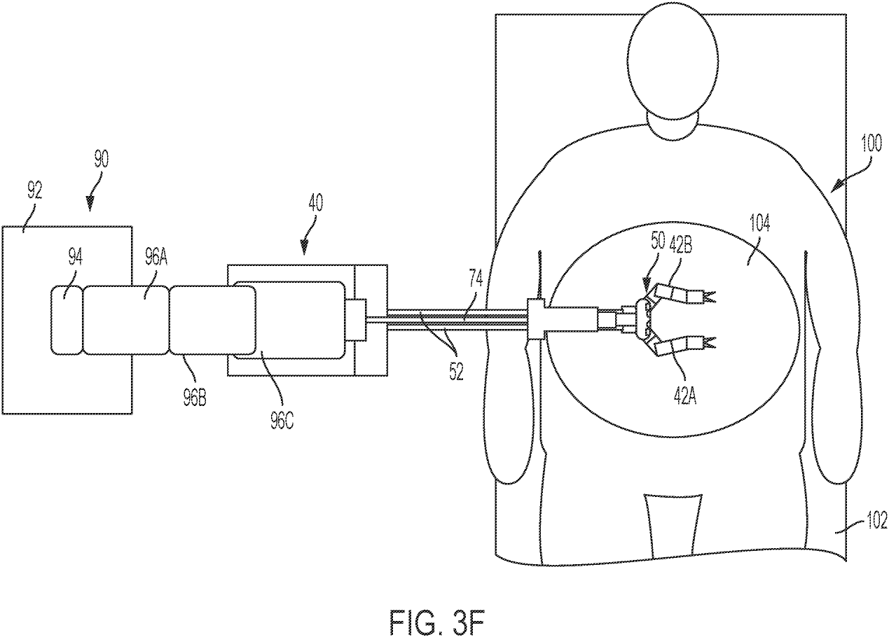

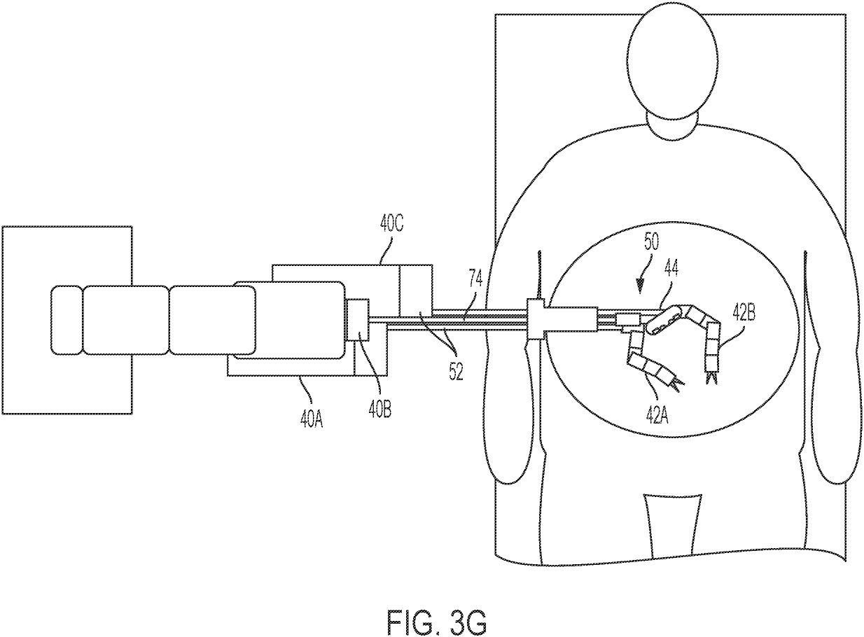

As shown in FIGS. 3A-3G, the motor unit 40 can be coupled to a support stanchion 90 that forms part of the robot support system (RSS), which in turn forms part of the surgical robotic system 10 of the present invention. The RSS is configured so as to mechanically move the motor elements positioned external to the body cavity of the patient, such that any motions occur about or relative to a trocar 108. The RSS thus provides for movement in the yaw, pitch and in some embodiments roll directions about the trocar so that during operation those degrees of freedom can be provided or conveyed to the robot arm subassemblies and to the camera subassembly without causing undo harm to the patient. This sort of motion can also be provided for by robotic coordination of multiple elements or can be enforced through the mode of articulation of the joints of the robotic arms. The illustrated support stanchion 90 can have any selected shape and size and is preferably configured to be able to move and manipulate the one or more components of the robotic unit 50 portion of the robotic subsystem 20. The support stanchion 90 can have a main body having a base element 92 and a vertically extending support beam 94 coupled thereto. The support beam 94 can be employed to provide mechanical support for a set of adjustment elements 96 that are coupled thereto. The adjustment elements 96 can be pivotably movable relative to each other via a pivot j oint. Those of ordinary skill in the art will readily recognize that the support stanchion 90 can employ one or more adjustment elements, preferably two or more adjustment elements, and most preferably three or more adjustment elements. In the illustrated embodiment, the adjustment elements 96 can include a first adjustment element 96A that is pivotably coupled to the support beam 94 via a first or proximal pivot joint 98A. The pivot joint can employ known assemblages of mechanical elements that allow for pivoting movement of the first adjustment element 96A relative to the support beam 94. The support stanchion 90 can also employ a second or middle adjustment element 96B that is coupled to the first adjustment element 96A by a second or middle pivot joint 98B. The second pivot joint 98B allows pivoting movement of the second adjustment element 96B relative to the first adjustment element 96A. The support stanchion 90 also employs a third or distal adjustment element 96C that is coupled to the second adjustment element 96B by a third or distal pivot joint 98C. The third pivot joint 98C allows pivoting movement of the third adjustment element 96C relative to the second adjustment element 96B.

The third or distal adjustment element 96C can also be coupled to the motor unit 40 via any selected mechanical connection, for translating or linearly moving the motor unit. The motor unit 40 can employ one or more drive elements or motor elements 40A-40C for driving one or more components of the robotic subsystem 20, and specifically for driving the robot arm subassembly 56 and the camera subassembly 78. Specifically, the support stanchion 90 can be configured for moving and adjusting one or more motor elements of the motor unit 40 in at least two degrees of freedom, and more typically in five or six degrees of freedom. In one embodiment, the motor unit 40 can be attached to the adjustment element 96C for adjusting the position of the motors 40A-40C and hence the position of one or more components of the robotic unit that is coupled to the motors. The linear or translational position of the motors can be adjusted by cooperative movement of one or more of the adjustment elements 96A-96C relative to each other via the pivot joints 98A-98C. Further, the motor elements can also be translationally moved relative to the third adjustment element 96C by sliding translational movement. This translation movement enables the depth of each motor element relative to the trocar to be able to be controlled independently of each other. In one embodiment, there exists between the third adjustment element 96C and each of the motor elements a linear degree of freedom typically in the form of a linear rail that allows each motor element to be controlled translationally relative to the trocar. The linear rails can exist between different motor elements of the motor unit 40. For example there can be a linear rail connecting the third adjustment element 96C to the camera motor element upon which there is a second and third linear rail that each connects respectively to the first and second robot arm motor elements.

Further, the position of the motors 40A-40C can be adjusted in the axial direction relative to the patient or can be moved in an arc like manner or can be moved in the vertical direction. In one embodiment, multiple motors 40A-40C can be attached to the same adjustment element 96C for simultaneously adjusting the position of the motors and hence the position of one or more components of the robotic unit that is coupled to the motors, as shown for example in FIGS. 3C and 3E-3G. In other embodiments, each of the motors is mounted to a separate adjustable support element for providing independent adjustment of each motor. In still other embodiments, two or more motors can be mounted on a common support element and the remaining motors on separate support elements.

The illustrated support stanchion 90 can be configured to carry any necessary mechanical and electrical cables and connections. The support stanchion 90 can be coupled to or disposed in communication with the computing unit 18 so as to receive control signals therefrom. The motor unit 40 can be coupled to one or more motors 40A-40C, and the motor unit via the interface elements 54, 76 can translate or axially move the camera and robot arm subassemblies. The adjustment element 96C can be sized and configured to mount the appropriate sized motor unit 40.

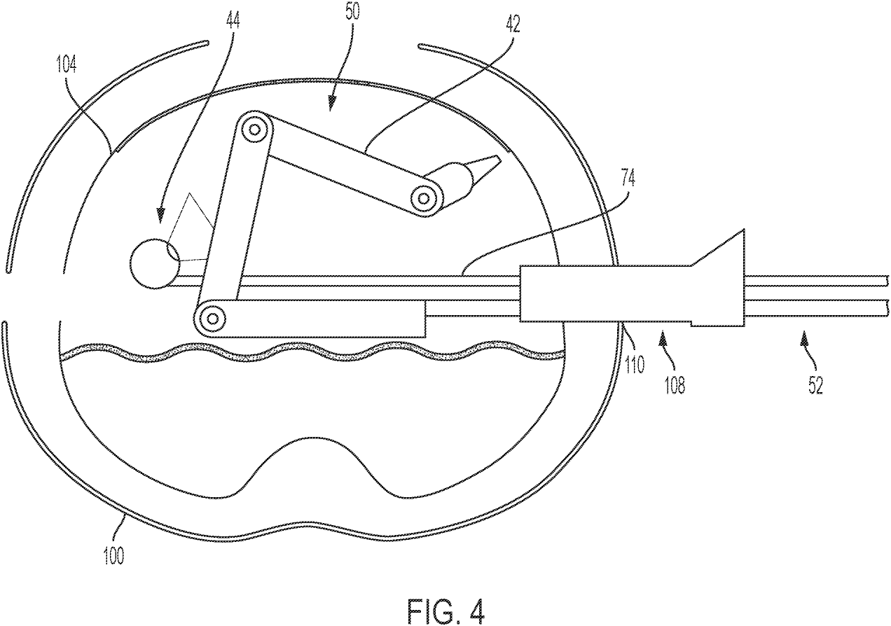

In use during surgery, a user, such as a surgeon, can setup the RSS in an operating room such that it is disposed in a location that is suitable for surgery and is positioned such that the support stanchion 90 and associated motor unit 40 is ready to be coupled to the robotic unit 50. More specifically, the motor elements 40A-40C of the motor unit 40 can be coupled to the camera subassembly 78 and to each of the robot arm subassemblies 56, 56. As shown in FIGS. 3A-3G and 4, the patient 100 is brought into an operating room and placed on a surgical table 102 and prepared for surgery. An incision is made in the patient 100 so as to gain access to a body cavity 104. A trocar device 108, or any similar device, is then inserted into the patient 100 at a selected location to provide access to the desired body cavity 104 or operation site. For example, in order to access a patient’s abdominal cavity, the trocar 108 may be inserted into and through the patient’s abdominal wall. In this example, the patient’s abdomen is then insufflated with a suitable insufflating gas, such as carbon dioxide. When the patient’s abdomen is properly insufflated, the RSS, including the support stanchion 90, can then be maneuvered into position over the patient 100 and the trocar 108. The camera subassembly 78 and one or more robot arm subassemblies 56 can be coupled to the motor unit 40 and can be inserted into the trocar 108 and hence into the body cavity 104 of the patient 100. Specifically, the camera assembly 44 and the robot arms 42A, 42B can be inserted individually and sequentially into the patient 100 through the trocar 108. The sequential insertion method has the advantage of supporting smaller trocars and thus smaller incisions can be made in patient, thus reducing the trauma experienced by the patient. Furthermore, the camera assembly 44 and the robot arms 42A, 42B can be inserted in any order or in a specific order. According to one practice, the camera assembly can be followed by the first robot arm and then followed by the second robot arm, all of which can be inserted into the trocar 108 and hence into the body cavity 104.

Once inserted into the patient 100, each component (e.g., the robot arms and the camera assembly) of the robotic unit 50 can be moved to a surgery ready position either at the direction of the surgeon or in an automated fashion. In some embodiments, the camera assembly 44 can employ stereoscopic cameras and can be configured such to be positioned equidistant from a shoulder joint of each robotic arm 42A, 42B and is thus centered therebetween. The alignment of the cameras 70A, 70B and the two shoulder joints forms the virtual shoulder of the robotic unit 50. The robot arms have at least six degrees of freedom, and the camera assembly has at least two degrees of freedom, thus allowing the robot to face and work in selected directions, such as to the left, right, straight ahead, and in a reverse position as described in further detail below.

Once inside the patient 100, the working ends of the robot arms 42A, 42B and the camera assembly 44 can be positioned through a combination of movements of the adjustment elements 96A-96C, the motors elements 40A-40C, as well as the internal movements of the articulating joints or sections 58 of the robot arms and the camera assembly. The articulating sections 58 allow the working ends of the robot arms 42 and the camera assembly 44 to be positioned and oriented within the body cavity 104. In one embodiment, the articulating sections provide for multiple degrees of freedom inside the patient, including for example movement in the yaw direction, the pitch direction, and the roll direction about the vertical shoulders of the robot arms. Further, movement in the yaw direction about the trocar 108 effectively translates the working ends of the robot arms to the left or to the right in the body cavity 104 relative to the trocar 108. Also, movement in the pitch direction about the trocar 108 effectively translates the working ends inside the patient up or down or into a reverse position. The motor elements, which can be moved or translated in an axial or linear manner to provide a translational degree of freedom, allows each working end to be inserted shallower or deeper into the patient along the long axis of the trocar 108. Finally, the articulating joints allow for small, dexterous motions and delicate manipulation of tissue or other tasks via the end-effector 64. For example, in one embodiment, the three articulating joints associated with the camera assembly 44 allow the associated imaging elements to be positioned in the most advantageous position for viewing the manipulation or other desired elements of the surgery. In combination, the three articulating joints enable the surgeon to yaw and pitch to any desired viewing angle and to adjust the angle of the apparent horizon. The combination of the capabilities of the different elements and different motions produces a system that is highly dexterous within a very large volume and which gives the device and the user a high degree of freedom of how to approach the work site and perform work therein. According to another embodiment, each robot arm and camera assembly can be inserted through their own independent trocars and are triangulated internally so as to perform work at a common surgical site.

The robotic subsystem 20 of the present invention provides for maximum flexibility of the device during surgery. The surgeon can operate the robot arms 42 and the camera assembly 44 at different surgical locations within the abdominal cavity 104 through a single point of incision. The surgical sites can include those sites to the left of the trocar insertion point, to the right of the trocar insertion point, ahead or in front of the trocar insertion point, and if needed behind the camera assembly 44 and viewing “back” towards the trocar insertion point. The robotic unit 50 of the present invention allows the surgeon to reverse the orientation of the robotic arms 42 and the camera assembly 44 viewpoint so as to view those portions of the abdominal cavity that lie behind the robotic unit 50 when inserted within the cavity 104. That is, the viewpoint of the camera robot assembly can be reversed so as to be backward facing. Likewise, the positions of the robot arms 42 can be reversed based on the multiple degrees of freedom of movement of the arms. By having a surgical robotic unit 50 that is capable of operating while facing towards the trocar insertion site greatly increases the flexibility of the overall surgical system 10, since the robotic unit 50 can reach anywhere within the abdominal cavity 104. With complete reach, the robotic unit 50 is able to perform any operation with only a single insertion site, which reduces patient trauma. A robotic unit that can reach and view the insertion site can also stitch closed the incision point, which would save time and tool usage in the operating room environment. Further, similar capabilities exist with regard to the robot arms, which can have at least six degrees of freedom internally plus any degree of freedom associated with the end-effector.

FIG. 4 is a general schematic representation of the robotic unit 50 of the present invention disposed within an abdominal cavity 104 of a patient 100 where the robotic unit is disposed in a backward facing orientation or position. The robotic unit 50 is passed through the trocar 108 that is inserted through the incision point 110 and into the cavity 104. As shown, the camera assembly 44 and the robot arms 42 are disposed in a back facing orientation according to the teachings of the present invention. The support stanchion 90, the robot arms 42 and the camera assembly 44 can be controlled by the computing unit 18 to perform or execute a combination of movements, such as movements or rotations in the axial, pitch, roll and/or yaw directions, that position the robot arms and camera assembly so as to face backwards towards the incision point 110 with a sufficiently clear view to perform surgical procedures.

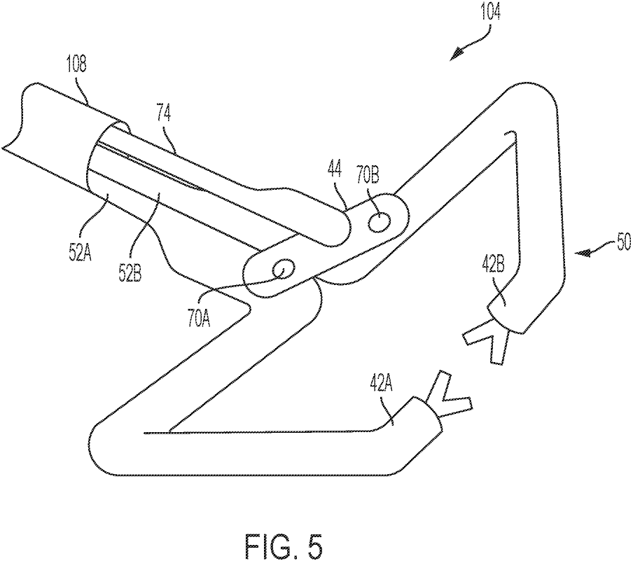

FIG. 5 is a schematic representation of the robotic unit 50 of the present invention when initially inserted through the trocar 108 into a body cavity 104, such as the abdominal cavity, of the patient. The illustrated positioning of the robot arms 42A, 42B and the camera assembly 44 forms a typical or normal operating position of the robotic unit 50 indicating that the unit is ready for use. The robotic unit 50 includes a pair of robot arms 42A, 42B, each of which is coupled to a corresponding support member 52A, 52B, respectively, that extends along a longitudinal axis. The robot arms 42A, 42B are movable relative to the support members 52A, 52B in multiple different directions and orientations and have corresponding shoulder joints, elbow joints, and wrist joints. The illustrated robot arms are identical and include a first robot arm 42A that can correspond to, as shown, a right robot arm and a second robot arm 42B that can correspond to, as shown, a left robot arm. The robotic unit 50 also includes a camera assembly 44 that employs a pair of stereoscopic cameras 70A, 70B formed by a pair of axially spaced apart lens systems and which, in the illustrated position, correspond to a right camera element 70A (e.g., a right eye) and a left camera element 70B (e.g., a left eye). The camera assembly 44 is mounted to a corresponding support member 74 and is movable relative thereto in multiple different directions, including the yaw, pitch, and roll directions. The camera assembly can be coupled to the support member using any selected mechanical connection that allows the assembly to move in multiple different directions, including the yaw and pitch directions. Each of the individual robot arms 42A, 42B and the camera assembly 44 are inserted through the trocar 108 at the incision point 110 and into the cavity 104, and are supported by their respective support members that extend along a longitudinal axis.

The robot arms 42A, 42B and the camera assembly 44 can be manipulated by a user, such as a surgeon, during use. If the user desires to position the robotic unit 50 in a backward facing (e.g., reverse) orientation or position so as to view the incision point 110 or other portions of the body cavity, then the robot arms 42A, 42B and the camera assembly 44 can be independently manipulated in a number of coordinated movements so as to move the respective components into the backward facing position. This can be achieved by a variety of different movements of the robot arms and the camera assembly. For example, the sensing and tracking unit 16, 16A can sense movement of the surgeon and generate signals that are received and processed by the computing unit 18. The computing unit in response can generate control signals that control movement of the robot arm subassembly and the camera subassembly. Specifically, movement of the hands and head of the user are sensed and tracked by the sensing and tracking unit 16, 16A and are processed by the computing unit 18. The control unit 26 can generate control signals that are conveyed to the robotic subsystem 20. In response, the motor unit 40, which includes one or more motors or drive elements, can be controlled to drive or move the camera subassembly 78 and the robot arm subassemblies 56, 56 in selected ways.

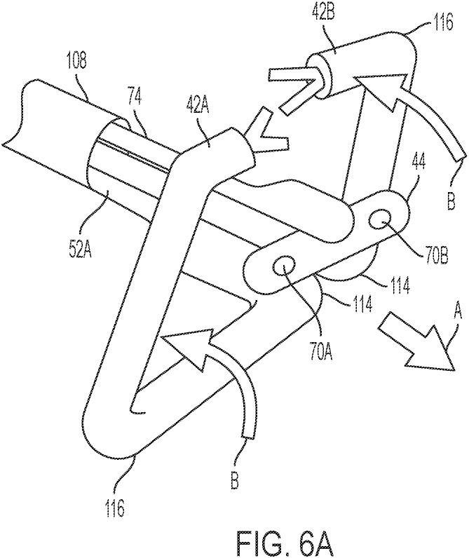

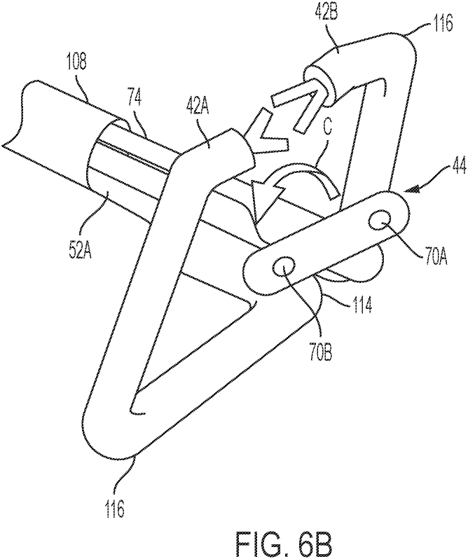

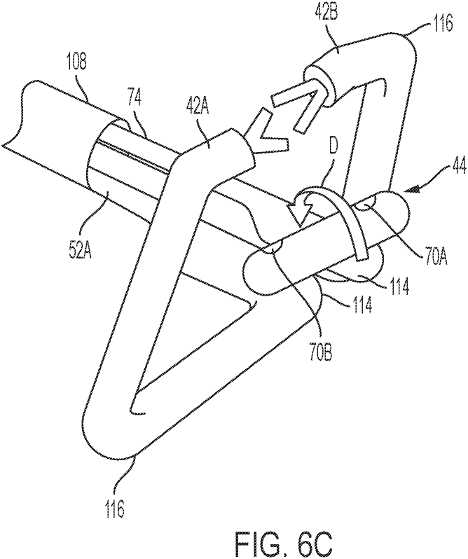

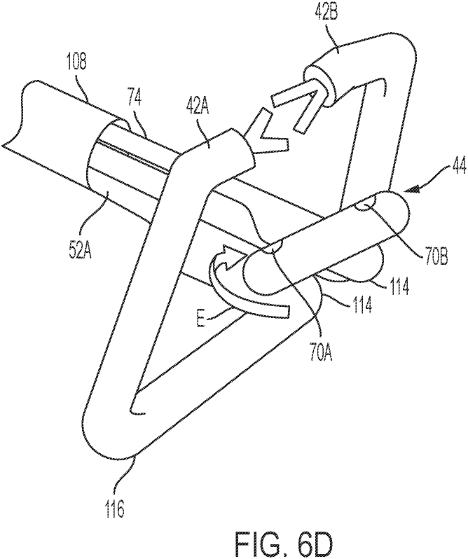

For example, if the user desires to position the robotic unit 50 into a backward facing position, the controller can generate and transmit appropriate instructions to the robotic subsystem to perform a series of coordinated movements, as shown for example in FIGS. 6A-6D. Initially, the camera support member 74 is moved in the axial direction away from the incision point 110 by for example one or more of the motor units and/or cooperative and coordinated movement of one or more of the adjustment elements 96 of the support stanchion 90. away from the incision point 110, as represented by arrow A in FIG. 6A. The axial movement of the support member 74 positions the camera assembly 44 in such a manner so as to be sufficiently clear of the robot arms to allow rotation of the arms without unwanted interference from the camera assembly. Enabled by the six degrees of freedom, the robot arms 42A, 42B can then be rotated upwardly and backward a selected amount at an elbow joint 116 and at a shoulder joint 114 formed by the corresponding articulation sections 58 of the robot arms, as indicated by arrows B. In the new orientation and position, the right robot arm 42A effectively becomes the left robot arm, and the left robot arm 42B effectively becomes the right robot arm. The support stanchion 90 can employ the motor assembly 40, which can in response to suitable control signals, move the camera assembly 44 and associated camera support member 74 in multiple different directions. The camera support element 74 can then be rotated or moved in a roll direction, as indicated by arrow C in FIG. 6B. In this orientation, the camera assembly 44 is rotated as well such that the camera elements effectively switch sides. For example, the camera element 70A is now disposed on the opposed side of the camera assembly 44, while concomitantly still functioning as the “right eye” of the camera assembly. Similarly, the camera 70B is now disposed on the opposed side of the camera assembly 44, while concomitantly still functioning as the “left eye” of the camera assembly. Further, in this position, the camera support member 74 is positioned out of the field of view of the camera elements 70A, 70B. The camera assembly 44 can then be rotated in the pitch direction about 180 degrees, as indicated by arrow D in FIG. 6C, such that the field of view of the cameras 70A, 70B are now facing rearward back towards the incision point 110.

According to an alternate practice, the camera support member 74 can be moved 180 degrees in the roll direction followed by a 180 degree rotation of the camera assembly 44 in the yaw direction, as indicated by arrow E in FIG. 6D. When the camera assembly 44 is rotated in this manner, the camera positions are reversed from the perspective of the user. Specifically, the left eye becomes the right eye, and the right eye becomes the left eye. This misplaced eye orientation can be addressed via the controller, which can swap instructions such that instructions intended for the left eye are now conveyed to the right eye and vice versa. According to another alternate practice, the camera support member 74 is initially disposed within the cavity so as to not obscure the insertion point 110 field of view from the perspective of the camera assembly, and as such only the camera needs to be moved or rotated. As such, the camera assembly 44 can be moved relative to the camera support member 74 either in a yaw direction or a pitch direction by about 180 degrees. When the camera assembly 44 is rotated, the arms are reversed from the perspective of the user. Specifically, the left arm becomes the right arm, and the right arm becomes the left arm. This misplaced orientation can be addressed via the controller, which can swap instructions such that instructions intended for the left arm are now conveyed to the right arm and vice versa. Specifically, a software correction or remap can be implemented so as to process commands for the correct robot arm and camera element (e.g., require the system software to remap the left controller to drive the right arm and vice versa). Further, the video feeds for the camera elements can be swapped so as to achieve the desired result.

In addition to movement of the camera assembly 44, the robot arms 42A, 42B can also be moved as well. For example, the robot arms 42A, 42B can be rotated to face backward towards the insertion point or site. The rotational movement of the robot arms can be accomplished by rotating the arms at the corresponding joints, such as for example at the shoulder joints 114, such that the arms rotate past the camera assembly and face backward towards the trocar 108.

According to still another practice, the camera assembly 44 can be inserted into the body cavity 104 of the patient 100 in the orientation shown in FIG. 6B (e.g., camera assembly disposed on top of the camera support member 74). If further movement of the camera assembly 44 is implemented, then the camera elements would be reversed relative to what is shown in FIGS. 6C and 6D.

Further, the user can position the robotic unit in a left facing mode, a right facing mode, an up facing mode and a down facing mode, through similar means of adjusting the relative angles of the joints of the arms and the camera, thereby enabling the user to operate at all angles relative to the insertion site. This can be further augmented by external yaw, pitch and roll of the motor elements to allow for translational placement and movement of the robotic unit within the body cavity.

An advantage of the above surgical robotic system 10 is that it is highly adaptable and maneuverable, and enables the surgeon to move the robotic unit 50 throughout the body cavity 104. Further, the robotic unit 50 can be oriented in many different ways and configurations, including viewing backwards toward the insertion point 110. Since the robotic unit 50 can reach and view the insertion point 110, the unit can also stitch closed the incision point 110, which saves time and tool usage in the operating room environment.

1. A surgical robotic system, comprising
a computing unit for receiving user generated movement data and for generating control signals in response thereto,
a robot support subsystem having a support stanchion, the support stanchion includes a base portion, a support beam having a first end coupled to the base and an opposed second end coupled to a proximal one of a plurality of adjustment elements, wherein the plurality of adjustment elements are arranged and disposed to form pivot joints between adjacent ones of the plurality of adjustment elements and between the proximal one adjustment element and the support beam, and
a robotic subsystem having
a motor unit having one or more motor elements associated therewith, wherein the motor unit is coupled to a distal one of the plurality of adjustment elements, and
a robotic unit having a camera subassembly and a plurality of robot arm subassemblies, wherein the camera subassembly and the plurality of robot arm subassemblies are coupled to the motor unit, and the motor unit when actuated moves one of the camera subassembly and the robot arm subassemblies in a selected direction,
wherein one or more of the plurality of adjustment elements and one or more of the camera subassembly and the robot arm subassemblies move in response to the control signals.

2. The surgical robotic system of claim 1, wherein the camera subassembly comprises
an axially extending support member,
an interface element coupled to one end of the support member, and
a camera assembly coupled to an opposed end of the support member.

3. The surgical robotic system of claim 2, wherein the interface element is configured for engaging with one or more of the motor elements of the motor unit.

4. The surgical robotic system of claim 3, wherein the camera assembly comprises a first camera element having a first light source associated therewith and a second camera element having a second light source associated therewith.

5. The surgical robotic system of claim 3, wherein each of the robot arm subassemblies comprises
an axially extending support member,
an interface element coupled to one end of the support member, and
a robot arm coupled to an opposed end of the support member.

6. The surgical robotic system of claim 5, wherein the motor unit includes a plurality of motor elements, and wherein each of the interface elements of the robot arm subassemblies is configured for engaging with different ones of the plurality of motor elements of the motor unit.

7. The surgical robotic system of claim 5, wherein the interface element of the camera subassembly is coupled to the same motor element as the interface element of one of the robot arm subassemblies.

8. The surgical robotic system of claim 5, wherein the motor unit includes a plurality of motor elements, and wherein the interface element of the camera subassembly and the interface element of one of the robot arm subassemblies are coupled to different ones of the plurality of motor elements.

9. The surgical robotic system of claim 5, wherein the robot arms have an end effector region, and wherein the camera assembly and the first and second robot arms can be sized and configured to be inserted into a cavity of a patient through an insertion point, and wherein the computing unit in response to the user generated control signals generates the control signals which are received by the first and second robot arms and the camera assembly for:
actuating each of the first and second robot arms so as to reverse direction such that the end effector region is facing towards the insertion point, and
moving the camera assembly in a selected direction such that the camera elements are facing towards the insertion point.

10. The surgical robotic system of claim 5, wherein the robot arms have an end effector region, and wherein the camera assembly and the first and second robot arms can be sized and configured to be inserted into a cavity of a patient through an insertion point, and wherein the computing unit in response to the user generated control signals generates the control signals which are received by the first and second robot arms and the camera assembly for
orienting the robot arms such that they face in a first direction that is transverse or orthogonal to an axis of the support member, and
actuating each of the first and second robot arms so as to reverse direction such that the end effector region is facing in a second direction that is substantially opposite the first direction.

11. The surgical robotic system of claim 5, wherein the robot arms have an end effector region, and wherein the camera assembly and the first and second robot arms can be sized and configured to be inserted into a cavity of a patient through an insertion point, and wherein the computing unit in response to the user generated control signals generates the control signals which are received by the first and second robot arms and the camera assembly for
orienting the robot arms such that they face in a first direction, and
actuating each of the first and second robot arms so as to reverse direction such that the end effector region is facing in a second direction that is substantially opposite the first direction.

12. The surgical robotic system of claim 9, further comprising, prior to moving the camera assembly towards the insertion point, rotating the camera support member so that the camera assembly is disposed above the camera support member and one or more camera elements of the camera assembly are facing away from the insertion point.

13. The surgical robotic system of claim 12, wherein the computing unit in response to the user generated control signals generates the control signals which are received by the first and second robot arms and the camera assembly for rotating the camera assembly in a pitch direction such that the camera elements are facing towards the insertion point.

14. The surgical robotic system of claim 12, wherein the computing unit in response to the user generated control signals generates the control signals which are received by the first and second robot arms and the camera assembly for rotating the camera assembly in a yaw direction such that the camera elements are facing towards the insertion point.

15. A method for moving a robotic unit in vivo, wherein the robotic unit includes a camera subassembly having a camera assembly coupled to a camera axially extending support member, a first robot arm subassembly having a first robot arm coupled to a first robot arm axially extending support member, and a second robot arm subassembly having a second robot arm axially extending support member, wherein when inserted in a cavity of a patient through an insertion point, the camera assembly and the first and second robot arms can be controlled for:
actuating at least one joint of each of the robot arms to reverse direction such that an end effector region of each of the first and second robot arms is facing towards the insertion point, and
moving the camera assembly in a selected direction such that the camera elements are facing towards the insertion point.

16. The method of claim 15, wherein the robotic unit is connected to a motor unit, further comprising actuating the motor unit so as to move the robotic unit or the camera assembly relative to the insertion site in a linear direction.

17. The method of claim 16, wherein the motor unit includes a plurality of motor elements, and wherein each of the interface elements of the first and second robot arm subassemblies is configured for engaging with different ones of the plurality of motor elements of the motor unit.

18. The method of claim 16, wherein the motor unit includes a plurality of motor elements, and wherein the interface element of the camera subassembly is coupled to the same motor element as the interface element of one of the first and second robot arm subassemblies.

19. The method of claim 16, wherein the motor unit includes a plurality of motor elements, and wherein the interface element of the camera subassembly and the interface element of one of the first and second robot arm subassemblies are coupled to different ones of the plurality of motor elements.

20. The method of claim 15, further comprising, prior to moving the camera assembly, rotating the camera support member so that the camera assembly is disposed above the camera support member and one or more camera elements of the camera assembly are facing away from the insertion point.

21. The method of claim 15, wherein the step of moving the camera assembly comprises rotating the camera assembly in a pitch direction such that the camera elements are facing towards the insertion point.

22. The method of claim 15, wherein the step of moving the camera assembly comprises rotating the camera assembly in a yaw direction such that the camera elements are facing towards the insertion point.

23. The method of claim 20, further comprising moving the camera support element in an axial direction away from the incision point prior to rotating the robot arms and the camera support assembly.

24. A method for moving a robotic unit in vivo, wherein the robotic unit includes a camera subassembly having a camera assembly coupled to a camera axially extending support member, a first robot arm subassembly having a first robot arm coupled to a first robot arm axially extending support member, and a second robot arm subassembly having a second robot arm coupled to an axially extending support member, wherein when inserted in a cavity of a patient through an insertion point, the camera assembly and the first and second robot arms can be controlled for:
actuating at least one joint on each of the first and second robot arms to reverse direction such that each an end-effector region of each of the first and second robot arms is facing in a direction that is orthogonal to an insertion axis, and
actuating at least one joint of the camera assembly to move the camera assembly in a selected direction such that the camera elements are facing in a direction orthogonal to the insertion axis.

25. The method of claim 24, wherein the robotic unit is connected to a motor unit, further comprising actuating the motor unit so as to move the robotic unit or the camera assembly relative to the insertion site.

26. The method of claim 25, wherein the motor unit includes a plurality of motor elements, and wherein each of the interface elements of the first and second robot arm subassemblies is configured for engaging with different ones of the plurality of motor elements of the motor unit.

27. The method of claim 25, wherein the motor unit includes a plurality of motor elements, and wherein the interface element of the camera subassembly is coupled to the same motor element as the interface element of one of the first and second robot arm subassemblies.

28. The method of claim 25, wherein the motor unit includes a plurality of motor elements, and wherein the interface element of the camera subassembly and the interface element of one of the first and second robot arm subassemblies are coupled to different ones of the plurality of motor elements.

29. The method of claim 24, further comprising, prior to moving the camera assembly, rotating the camera support member so that the camera assembly is disposed above the camera support member and one or more camera elements of the camera assembly are facing away from the reverse facing direction.

30. The method of claim 24, wherein the step of moving the camera assembly comprises rotating the camera assembly in a pitch direction such that the camera elements are facing in the reverse facing direction.

31. The method of claim 24, wherein the step of moving the camera assembly comprises rotating the camera assembly in a yaw direction such that the camera elements are facing in the reverse facing direction.

32. The method of claim 24, further comprising moving the camera support element in an axial direction away from the incision point prior to rotating the robot arms and the camera support assembly.
